(19) United States
(12) Patent Application Publication
(10) Pub. No.: US 2026/0061212 A1
(43) Pub. Date: Mar. 5, 2026

(54) ELECTROMAGNETIC RADIATION TREATMENT

(71) Applicants: Jacob Zabara, Miami Beach, FL (US); University of Miami, Miami, FL (US)

(72) Inventors: Jacob Zabara, Miami Beach, FL (US); Scott Welford, Miami, FL (US); John Chetley Ford, Miami, FL (US)

(21) Appl. No.: 19/285,328

(22) Filed: Jul. 30, 2025

Related U.S. Application Data
(63) Continuation of application No. PCT/US2024/014147, filed on Feb. 2, 2024.
(60) Provisional application No. 63/482,926, filed on Feb. 2, 2023.

(51) Int. Cl. A61N 2/00; A61N 2/02

(52) U.S. Cl. CPC A61N 2/002; A61N 2/02

(57) ABSTRACT

Described herein is a system for providing electromagnetic energy comprising a first plate configured to be positioned adjacent to ah area to be treated, a second plate configured to be positioned adjacent to the area to be treated and at a distance from the first plate, the second plate being opposite and not contacting the first plate, the second plate being configured to be substantially parallel to the first plate, an electromagnetic coil configured to receive electrical energy, a power source configured to supply electrical energy to the first plate, the second plate, and the electromagnetic coil.

### CROSS-REFERENCE TO RELATED APPLICATIONS

This application claims the benefit of U.S. Provisional Patent Application No. 63/482,926 filed Feb. 2, 2023 entitled “ELECTROMAGNETIC RADIATION TREATMENT”, which is incorporated by reference herein in its entirety.

### BACKGROUND OF THE INVENTION

Brain tumors can be divided into primary and metastatic categories. The incidence of central nervous system (CNS) metastases is greater than primary brain tumors. Meningiomas are highest followed by infiltrating gliomas of which glioblastoma (GBM) is the most prevalent. Lung cancer, breast cancer and melanoma frequently metastasize to the brain but any malignancy may metastasize to the brain. They are round in shape and usually a distinct border is present.

### SUMMARY

In certain embodiments, a system for providing electromagnetic energy is disclosed. The system comprising: a first plate configured to be positioned adjacent to an area to be treated; a second plate configured to be positioned adjacent to the area to be treated and at a distance from the first plate, the second plate being opposite and not contacting the first plate, the second plate being configured to be substantially parallel to the first plate; an electromagnetic coil configured to receive electrical energy; a power source configured to supply electrical energy to the first plate, the second plate, and the electromagnetic coil; and a controller electrically coupled to one or more of the first plate, the second plate, the electromagnetic coil, and the power source. In certain embodiments, the controller being configured to independently provide electrical energy such that: a first electromagnetic field is induced between the first plate and the second plate within the area to be treated. In certain embodiments, the first electromagnetic field has a frequency range of 50-900 kHz; and a second electromagnetic field is induced by the electromagnetic coil.

In certain embodiments, the system further comprising a spacing system, the spacing system being configured to position each of the first plate, the second plate, and the electromagnetic coil with respect to the area to be treated. In certain embodiments, the first electromagnetic field is defined by a frequency range of 100-300 kHz. In certain embodiments, the first electromagnetic field is defined by a frequency of 200 kHz. In certain embodiments, the first electromagnetic field is defined by a voltage range of 18-300 V.

In certain embodiments, the electromagnetic coil is configured and adapted to provide the second electromagnetic field at a frequency to induce resonance of ions or molecules of a cell, a neuron, or a membrane within the area to be treated. In certain embodiments, the electromagnetic coil is configured to provide the second electromagnetic field having a frequency range of 10-60 MHz. In certain embodiments, the electromagnetic coil is controlled independently from the first plate and the second plate. In certain embodiments, the electromagnetic coil defines a shape and a diameter. In certain embodiments, the electromagnetic coil is configured and adapted to change the shape or the diameter to treat the area to be treated. In certain embodiments, the electromagnetic coil is a Figure-8 coil.

In certain embodiments, a method of providing electromagnetic energy using the system is disclosed. In certain embodiments, the system comprising the steps of: a) providing the system such that the first plate is positioned adjacent to the area to be treated and the second plate is positioned adjacent to the area to be treated, the second plate being substantially parallel to the first plate, the second plate being positioned at a distance from the first plate such that the area to be treated is disposed therebetween and such that the second plate is not contacting the first plate; b) providing the electromagnetic coil near the area to be treated; and c) supplying electrical energy from the power source via the controller to the group consisting of: the first plate, the second plate, and the electromagnetic coil.

In certain embodiments, a first electromagnetic field is induced between the first plate and the second plate within the area to be treated. In certain embodiments, the first electromagnetic field has a frequency range of 50-900 kHz; and a second electromagnetic field is induced by the electromagnetic coil. In certain embodiments, the second electromagnetic field has a frequency range of 10-60 MHz. In certain embodiments, the first electromagnetic field is defined by a frequency range of 100-300 kHz. In certain embodiments, the first electromagnetic field is defined by a frequency range of 200 kHz.

In certain embodiments, the first electromagnetic field and the second electromagnetic field are provided for a cumulative duration selected from the group consisting of: 5 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 24 hours, 48 hours, and 72 hours.

In certain embodiments, the method further comprising the step of: (d) treating the area to be treated with ionizing radiation. In certain embodiments, the supplying of step (c) and the treating of step (d) are done simultaneously for up to 5 minutes. In certain embodiments, the method further comprising the step of: (e) treating the area to be treated with infrared energy. In certain embodiments, the supplying of step (c) and the treating of step (e) are done simultaneously for up to 15 minutes.

In certain embodiments, during step (a): the first plate is provided to a left section of the area to be treated; and the second plate is provided to a right section of the area to be treated. In certain embodiments, during step (b), the electromagnetic coil is provided to a frontal section of the area to be treated. In certain embodiments, step (c) includes supplying electrical energy with a first characteristic to the electromagnetic coil independently from the electrical energy with a second characteristic supplied to the first plate and second plate. In certain embodiments, the first characteristic is defined by a first frequency and a first power. In certain embodiments, the second characteristic is defined by a second frequency and a second power.

In certain embodiments, the method further comprising the step of: (f) configuring a shape or a diameter of the electromagnetic coil such that the electromagnetic coil is configured and adapted to treat the area to be treated. In certain embodiments, the configuring of step (f) changes a focus and an intensity of the second electromagnetic field.

### BRIEF DESCRIPTION OF THE DRAWINGS

For a fuller understanding of the nature of the present invention, reference should be had to the following detailed description taken in connection with the accompanying drawings in which:

FIG. 1A is a top-view schematic representation illustrative of one embodiment of a system for providing electromagnetic energy in accordance with the present disclosure;

FIG. 1B is a side-view schematic representation illustrative of one embodiment of a system for providing electromagnetic energy in accordance with the present disclosure;

FIGS. 2A-2B are graphical representations of data illustrating the efficacy of certain exemplary embodiments of the present disclosure; and

FIG. 3 is a flow diagram illustrative of one embodiment of a method for providing electromagnetic energy in accordance with the present disclosure.

Like reference numerals refer to like parts throughout the several views of the drawings.

### DETAILED DESCRIPTION

Brain tumors can be divided into primary and metastatic categories. The incidence of CNS metastases is greater than primary brain tumors. Meningiomas are highest followed by infiltrating gliomas, of which glioblastoma (GBM) is the most prevalent. Lung cancer, breast cancer and melanoma frequently metastasize to the brain, but any malignancy may metastasize to the brain. They are round in shape and usually a distinct border is present.

Embodiments or the present disclosure are directed towards the treatment of brain cancer. In one embodiment, the brain cancer is primary brain cancer such as astrocytoma, oligodendroglioma, neuroblastoma, medulloblastoma or ependydoma. In another embodiment, the brain cancer is a mixed glioma, for example, a malignant tumor that contains astrocytes and oligodendrocytes. In another embodiment, the brain cancer is glioma, including high-grade glioblastoma multiforme. In yet another embodiment, the glioma molecular subtype is proneural. In another embodiment, the brain cancer could include metastatic brain cancer.

Types of brain tumors and cancer are well known in the art. Glioma is a general name for tumors that arise from the glial (supportive) tissue of the brain. Gliomas are the most common primary brain tumors. Astrocytomas, ependymomas, oligodendrogliomas, and tumors with mixtures of two or more cell types, called mixed gliomas, are the most common gliomas. The following are other common types of brain tumors: Acoustic Neuroma (Neurilemmoma, Schwannoma. Neurinoma), Adenoma, Astracytoma, Low-Grade Astrocytoma, giant cell astrocytomas, Mid- and High-Grade Astrocytoma, Recurrent tumors, Brain Stem Glioma, Chordoma, Choroid Plexus Papilloma, CNS Lymphoma (Primary Malignant Lymphoma), Cysts, Dermoid cysts, Epidermoid cysts, Craniopharyngioma, Ependymoma Anaplastic ependymoma, Gangliocytoma (Ganglioneuroma), Ganglioglioma, Glioblastoma Multiforme (GBM), Malignant Astracytoma, Glioma, Hemangioblastoma, Inoperable Brain Tumors, Lymphoma, Medulloblastoma (MDL), Meningioma, Metastatic Brain Tumors, Mixed Glioma, Neurofibromatosis, Oligodendroglioma. Optic Nerve Glioma, Pineal Region Tumors, Pituitary Adenoma, PNET (Primitive Neuroectodermal Tumor), Spinal Tumors, Subependymoma, and Tuberous Sclerosis (Bourneville's Disease).

Existing therapies involve significant toxicities that include surgical morbidity, cerebral edema, radiation necrosis and radiation damage. The therapies are usually evaluated based on survival time. In the absence of metastatic growth, an alternative cure or treatment of the present disclosure can be applicable. With multiple brain metastases, radiation usually of the entire brain is performed.

It would be desirable to provide improved methods of treatment which prolongs survival and may even eliminate the glioblastoma cancer.

Certain embodiments of the present disclosure can prolong survival and can even eliminate the glioblastoma cancer. Such technology is presented herein where conductive plates (e.g., metal, such as copper, or similar material composition, or alloys of the same) are placed on either side of the head and do not require the head to be shaved. It does not require chemotherapy to be effective but can stand alone. Another technology presented herein is to couple the plate technology with electromagnetic coils, which is explained further herein. The plates and the coils act on two different mechanisms, which can summate and improve effectiveness of each one alone. The toxicities or clinical damage of these technologies is minimal or even completely absent, which would make them clinically desirable, especially in older patients.

Standard of treatment options are, for example, surgery, radiation treatment or chemotherapy either alone or together. An electromagnetic therapy as described herein would be more desirable in many circumstance as such electromagnetic therapy would not result in the undesirable toxicities present in the standard therapies. Standard therapies act by killing the cancer cells, but often also may destroy normal cells as a dangerous side effect. The plate and coil treatment, as described in connection with certain embodiments of the present disclosure, is in comparison, benign and apparently does not affect normal cells. The electromagnetic treatment of the present disclosure can be used either alone or in conjunction with any of the conventional cancer treatments known in the art.

It would be advantageous to apply an electromagnetic treatment (such as plate or coil treatments of certain embodiments of the present disclosure), which would act independently or improve standard treatment options. A major advantage of this novel form of treatment is that the treatment is effective when acting independently of standard treatment resulting in the virtual absence of toxic or damaging effects. Presently, with standard treatments, there is much discussion about what type or combination of treatments would be efficacious or desirable.

The standard treatment of glioblastoma, as is standard for many forms of cancer, is surgery followed by radiation and chemotherapy. Survival is only about twelve to fifteen weeks. Glioblastoma is very disabling in that it disrupts normal brain function and causes seizures.

Radiation and chemotherapy act by destroying cancer cells, but often also kill normal cells. Intensity of dose has to be carefully evaluated to diminish as much as possible the damage to normal cells. There are also incapacitating side effects such as nausea and vomiting.

Electromagnetic fields are a relatively safe way of curing or preventing cancer and also could also be used with standard treatments to improve their efficacy. Such electromagnetic fields are non-invasive and improve the efficacy of standard treatments.

Experiments are underway on this GBM cell culture model to determine optimum parameters of EMF and radiation. Experiments with EMF using copper plates surrounding the GBM culture have shown initial results. Cells were exposed to EMF for up to 72 hours. Also there were experiments involving ionizing radiation.

EMF significantly reduced GBM in initial experiments. Immediately after EMF, these cells were treated with ionizing radiation (low grade) which further reduced GBM. These results showed that EMF by itself can significantly reduce GBM. In addition, the combination of ionizing radiation and EMF is actually synergistic. The action of ionizing radiation by itself (which is standard therapy) shows significant reduction of GBM in these experiments. When ionizing radiation is combined with EMF, the reduction of GBM is even greater showing that the combination is synergistic. However, the safety factor in EMF alone as a therapy is much better than ionizing radiation, chemotherapy or surgery.

Certain embodiments of the present disclosure include a system for providing electromagnetic energy (or treating cancer) includes a first plate (positioned adjacent to an area to be treated), a second plate (positioned adjacent to the area to be treated and substantially parallel to the first plate); and an electromagnetic coil configured to receive electrical energy. Such a system can also include a power source configured to supply electrical energy to the first plate, the second plate, and the electromagnetic coil. Such a system can also include a controller electrically coupled to the first plate, the second plate, the electromagnetic coil, and/or the power source. The controller can be being configured to provide electrical energy such that: a first electromagnetic field (with a frequency in the range of 50-900 kilohertz (“kHz”)) is induced between the first plate and the second plate within the area to be treated; and a second electromagnetic field is induced by the electromagnetic coil.

In at least one embodiment of the present invention, the area to be treated or target area includes a plurality of target area cells. In at least one embodiment, the method also includes isolating the target area; however, it is envisioned that in at least some applications, the target area will include the entire body of the patient, in which case, isolation is not required.

The present method can also include selecting a source of electromagnetic radiation, for example, low-frequency electromagnetic radiation and/or radio frequency electromagnetic radiation. Further, the method can include selecting treatment parameters for an electromagnetic radiation treatment session. The treatment parameters can include, but are not limited to, a pulse frequency of the electromagnetic radiation, the pulse duration of the electromagnetic radiation, an electrical current, a magnetic field density, and a treatment-session exposure time.

The electromagnetic radiation treatment regimen can also include initiating the electromagnetic radiation treatment session, and applying an amount of electromagnetic radiation from the selected electromagnetic radiation source(s) to the target area in accordance with the treatment parameters selected.

Additionally, the present method can further include terminating the electromagnetic radiation treatment session. After the session is terminated, the present method can provide for measuring a response of at least some of the plurality of target area cells to the electromagnetic radiation treatment session, and evaluating the response of the plurality of target area cells measured to the electromagnetic radiation treatment session. Based upon the response of the target area cells measured, revised treatment parameters may be selected, and one or more subsequent treatment session may be conducted.

These and other objects, features and advantages of the present invention will become clearer when the drawings as well as the detailed description are taken into consideration.

The treatment regimen of the present disclosure can utilize electromagnetic radiation (“EMR”) to treat cells or tissues, such as cancerous cells. Certain embodiments of the present disclosure utilize both electrical current and magnetic fields as significant vehicles of action on specific molecular and atomic components of cancerous cells, such as, the protons and electrons of the cancerous cells, to elicit a beneficial therapeutic effect. The present method takes advantage of the significant differences in the electromagnetic properties of normal and cancerous cells or tissues. Specifically, the differences between the electromagnetic properties of normal cells, cancerous cells, and virally-infected cells transcend the molecular realm, and extend into the quantum mechanism realm. While the present EMR treatment methodology is disclosed hereinafter with a primary focus on the treatment of cancerous cells, it is understood to be within the scope and intent of the present disclosure to apply the present EMR treatment regimen to other abnormal cells including, but not limited to, cells affected by various cancers, including sarcoma and brain tumors.

The method of the present disclosure provides a significantly greater safety profile than the previously known methods of treating cancer cells, such as, surgery, chemoradiation, and/or pharmaceutical treatment regimens. The safety of the present method inures from the electrodynamic differences between the cancerous cell and the normal cell (e.g., the metabolic rate of the cancerous cell is much greater than a normal cell, which plays a role in the safety profile of the present devices and methods of the present disclosure).

Further, the present treatment regimen can be used to treat abnormal cells associated with the at least the following types of viral infections infections caused by several oncologic viruses may be treated or ameliorated by the use of EMR treatment. EMR treatment may be used for treatment infections by several oncogenic viruses including Epstein-Barr virus, human papilloma virus, Kaposi's sarcoma-associated herpes virus, herpes viruses, hepatitis B and C, human T-lymphotropic 1. In addition, EMR treatment may be used to treat several viral-induced neurological disorders, including but not limited to polio, meningitis, encephalitis and rabies.

### Cellular Control Systems

The control system of a cell is designated as one of three types: (1) bioproportional control, (2) bioderivative control and (3) biointergral control, each indicative of the operation of the control system in physical and electrical analysis. Bioproportional control is present in a cancerous cell throughout its reproductive activity, and allows for significant changes in a normal cell as it transforms into a cancerous cell. This is not a statistical series of events based on randomness, but, rather, directed events occurring in a specific sequence which determine the survival and development of a cancer cell. Bioderivative control regulates cell division and its underlying DNA processes. In cancerous cells, bioderivative control is present and regulates the initiation, or beginning phase, but not the progression, or final phase. Biointergral control addresses small errors that occur repeatedly over time. Implementation of the present electromagnetic radiation (“EMR”) treatment regimen introduces deliberate errors into the control system(s) of a cancerous cell to counteract the cell's reproductive and survival functions, as discussed in greater detail hereinafter.

### Low-Frequency (“LF”) Electromagnetic Radiation Treatment

Low-frequency (“LF”) electromagnetic radiation is utilized in the present EMR treatment regimen to produce a pulsed magnetic field of low frequency in a range of about 0.5 to 200 Hertz (“Hz”) resulting in a pulsed electric field. In some embodiments, the low frequency is in a range of about 1 Hz to about 5 Hz, about 5 Hz to about 10 Hz, about 10 Hz to about 15 Hz, about 15 Hz to about 20 Hz, about 20 Hz to about 25 Hz, about 25 Hz to about 30 Hz, about 30 Hz to about 35 Hz, about 35 Hz to about 40 Hz, about 40 Hz to about 45 Hz, about 45 Hz to about 50 Hz, about 50 Hz to about 55 Hz, about 55 Hz to about 60 Hz, about 60 Hz to about 65 Hz, about 65 Hz to about 70 Hz, about 70 Hz to about 75 Hz, about 75 Hz to about 80 Hz, about 80 Hz to about 85 Hz, about 85 Hz to about 90 Hz, about 90 Hz to about 95 Hz, about 95 Hz to about 100 Hz, about 100 Hz to about 105 Hz, about 105 Hz to about 110 Hz, about 110 Hz to about 115 Hz, about 115 Hz to about 120 Hz, about 120 Hz to about 125 Hz, about 125 Hz to about 130 Hz, about 130 Hz to about 135 Hz, about 135 Hz to about 140 Hz, about 140 Hz to about 145 Hz, about 145 Hz to about 150 Hz, about 150 Hz to about 155 Hz, about 155 Hz to about 160 Hz, about 160 Hz to about 165 Hz, about 165 Hz to about 170 Hz, about 170 Hz to about 175 Hz, about 175 Hz to about 180 Hz, about 180 Hz to about 185 Hz, about 185 Hz to about 190 Hz, about 190 Hz to about 195 Hz, about 195 Hz to about 200 Hz.

The pulsed electric field results in an alternating current acting upon the critical membranes of the cell, most directly on the cellular membrane. The LF electromagnetic radiation is achieved through the use of LF coils, which are usually circular or double constructed and are designed for to apply LF electromagnetic radiation to preselected regions of the body in a similar manner as the RF coils discussed hereinafter. The main effect of the application of LF electromagnetic radiation is to change the polarization of the membranes of target cancerous cells.

The membrane polarization change is a function of the intensity, frequency, and direction of the alternating current generated by the magnetic field described by the following hyperbolic relationship:

$$\frac{dB}{dt} = C \cdot R \frac{1 + D}{TC} \qquad ( 1 )$$

Where

$$\frac{dB}{dt}$$

is the rate of change of the magnetic field (B), D is the time for a magnetic pulse to reach maximum, and TC is the infected tissue's electrical time constant, which is a function of the dielectric, resistive, and magnetic properties of the infected tissue. C is a constant for the tissue radius and the magnetic field orientation.

Activation of protons by an appropriate magnetic field can increase the membrane potential and augment ATP synthesis, producing increased energy for the cell. Conversely, deactivation of protons by an appropriately oriented magnetic field can decrease or prevent ATP synthesis, and thus significantly diminish the energy available to a virally infected cell.

The specific pathway(s) of electrical current through infected tissue is difficult to predict, since the tissue segment contains blood vessels, connective tissue, etc. However, the present EMR treatment focuses the positive polarity of the electric field within the cancerous tissue and the opposite, negative polarity of the electric field outside the virally infected cell to affect the membrane potential. For example, to decrease activity of the membrane, the electric field has a direction of positive polarity within the cancerous tissue and negative polarity outside the cancerous tissue, resulting in a hyperpolarization of the membrane. The frequency, intensity, and direction of the magnetic field will be selected and adjusted based upon the nature and type of target cancerous tissue.

### Radio-Frequency (“RF”) Electromagnetic Radiation Treatment

In embodiments of the present disclosure, the EMR treatment regimen includes applying radio-frequency (“RF”) electromagnetic radiation to target cancerous cells, in order to produce a direct magnetic field effect on nuclei and electrons in the cancerous tissue.

A radio-frequency electromagnetic radiation coil can be utilized both to supply electromagnetic radiation to affect the nuclei and electrons of a target cell, as well as to detect nuclear or atomic magnetic signals generated in the cancerous tissue. A receiver can be utilized to demodulate the significant signal from the carrier frequency. The radio frequency pulses can be generated with center frequencies, bandwidths, amplitudes and phases being preselected based upon the type of cancerous tissue being treated, and its location with the patient's body. The bandwidth can be selected to correspond to the thickness of the cancerous tissue. The duration or shape of the radio frequency pulse relates to the bandwidth, while the amplitude of the radio frequency pulse determines the intensity of the magnetic field. In at least one embodiment, the radio frequency pulse envelope can be produced by digital means.

In at least one embodiment of the present EMR treatment regimen wherein RF electromagnetic radiation is applied, RF electromagnetic radiation coils are arranged substantially parallel to an axis of the patient. For example, in some aspects, the coils include an axis of orientation is that substantially the same as axis of orientation of the patient's body being treated. The coils may be resistive, or superconducting, such as, liquid helium cooled coils. Implementation of the EMR treatment regimen in accordance with the present disclosure may require utilization of one or more of a variety of RF electromagnetic radiation coils, including (1) head coil, (2) integral body coil, (3) spine coil, (4) neck coil, (5) abdominal coil, (6) chest coil, (7) knee coil, (8) shoulder coil, (9) flexible coils, (10) temporomandibular coil, etc., and combinations thereof. The RF electromagnetic coils may surround the entire body or only part of the body, or may be placed next to body or body part. Magnetic fields can be developed by utilizing at least one coil, or several sets of coils, one for each spatial dimension, or functional initiative. The RF electromagnetic radiation coils may be used individually, collectively, or in groups. Moreover, directed windings are oriented in three orthogonal directions. RF electromagnetic radiation pulses or waves are applied repeatedly in a regulated pulse or wave sequence.

The therapeutic effect of the present EMR treatment is a function of the energy imparted by the magnetic field produced via the RF electromagnetic radiation. More specifically, the RF electromagnetic radiation excites the atoms or nuclei of the target cells. The duration of excitation of the atoms or nuclei can be determined by reorienting them under a steady magnetic field by a radio frequency pulse. For instance, in a steady magnetic field, a 90° radio frequency pulse excites the nuclei and pushes the oriented nuclei into the transverse, or perpendicular, plane. The nuclei then return to their original positions generated by the steady magnetic field, called relaxing, which has a characteristic time constant that defines the duration of excitation. The time constant is different for cancerous tissue than for other normal tissues of the body. It also varies for different tissues. Fluids have a relatively long time constant in a range of about 1500-2000 ms, water-containing tissues have a time constant in a range of about 400-1200 ms, and fatty tissues have a relatively short time constant in a range of about 100-500 ms.

By implementing the present EMR treatment regimen, it is possible to selectively target protons (H*) of protein, fat, carbohydrate, protein-bound water, or bulk water. A frequency selective for RF electromagnetic radiation excitation is applied for each of the above entities, and can be applied for all of them at the same time or in sequence. For example, RF electromagnetic radiation can be used to activate fat or water by selection of the appropriate corresponding frequency. The frequency can be determined by the Larmor equation. For instance, water protons precess 220 Hz faster than fat protons when exposed to a magnetic flux density of about at 1.5 Tesla (“T”). Protons in protein-bound water have a resonant frequency which is about 500 to 2500 Hz different from bulk-water proton frequency. However, since protein-bound protons and bulk-water protons are in rapid exchange, the excitation can be quickly transferred from protein-bound water to bulk water. Notably, these excitation times are increased in the case of cancerous cells.

In certain embodiments, the present EMR treatment regimen comprises enhancing or doping target tissues, such as virally infected cells or tissues, with gadolinium, to increase the precessional frequency of protons. Other chemicals may also be utilized to enhance or dope target tissues. For example, xenon gas may be used to enhance spin effects by producing xenon in a hyperpolarized form, and causing an increased magnetization. The hyperpolarization can also be produced by rubidium vapor. For example, xenon is soluble in blood and has a high affinity for lipids. Rubidium vapor can be excited by a diode laser, and act on the electrons. After electronic polarization, the xenon nuclei are excited by contact with the rubidium atoms, and then cooled to xenon ice.

In certain embodiments, photons are utilized to augment the effect of electromagnetic radiation on electrons and protons. In one embodiment, a radioactive agent or pharmaceutical, such as technetium (Tc-99), which produces high-energy photons, e.g., 140 kilo electron volt photons, is utilized. Technetium has a half-life of about six hours and can be attached to a molecule which combines with a cancerous cell. For instance, in one embodiment, Tc-99 methylene diphosphonate (MDP) can be injected intravenously and absorbed by, and bound to, bone as a result of osteoblastic activity caused by metastatic deposition.

Moreover, in at least one embodiment, application of RF electromagnetic radiation is repeated at suitable intervals to maintain the excitation for a considerable period of time. The Larmor frequency (r/2πB) for excitation of protons (H⁺) increases with an increase of the magnetic field as shown in Table 1 herein.

Larmor frequency (r/2πB), which relates to the angular momentum of a spinning or precessing proton, for excitation of a proton (H⁺) increases with an increase of the magnetic field, or magnetic flux density, as shown in Table 1.

TABLE 1
Larmor Frequency for Varying Magnetic Flux Densities

| Magnetic Flux Density | Larmor Frequency | |
|---|---|---|
| 0.15T | 6.39 | MHz |
| 0.5T | 21.29 | MHz |
| 1.5T | 63.87 | MHz |
| 3.0T | 127.74 | MHz |

Cancerous cells have increased excitation times compared to normal cells. Thus, the excitation duration of protons is appreciably greater when the proton is located in or within the immediate environment of a cancerous cell. This indicates either an increased binding of the proton, or a decreased threshold, or a changed resonant frequency, in comparison to the normal cell. The cell's return to normal upon application of the present EMR treatment regimen can be observed by the cancerous cell's excitation time becoming essentially the same as the excitation time of a normal cell.

Accordingly, the excitation time can also be utilized to measure the progress of the EMR treatment regimen. For instance, the electromagnetic radiation fields generated by the proton are detected via receiver coils. Thus, almost simultaneously with application of the EMR treatment regimen of the present disclosure, it is possible to measure the real-time effect of the treatment.

### Combined LF and RF Electromagnetic Radiation Treatment

In at least one embodiment of the present EMR treatment regimen, LF electromagnetic radiation is applied in conjunction with RF electromagnetic radiation as discussed above. Further, in one embodiment, the RF electromagnetic radiation and LF electromagnetic radiation are applied in series, whereas, in at least one further embodiment, the RF electromagnetic radiation and LF electromagnetic radiation are applied in parallel (e.g., simultaneously). In at least one embodiment, LF electromagnetic radiation and RF electromagnetic radiation are applied in an alternating manner. In at least one embodiment, LF electromagnetic radiation and RF electromagnetic radiation are applied partially in parallel and partially in an alternating manner. In some aspects, the application of LF electromagnetic radiation and RF electromagnetic ration partially overlap. In at least one embodiment, LF electromagnetic radiation and RF electromagnetic radiation are applied in a series. Appropriately shaped electromagnetic radiation pulses can be generated at Larmor or other frequencies, and an alternating magnetic field of LF electromagnetic radiation is produced. With RF electromagnetic radiation, a steady magnetic field is employed.

A variety of known components may be arranged and utilized to produce the magnetic fields required for implementation of the present EMR treatment regimen to act on the cancerous tissue. For instance, a system can be used to generate and apply the required electromagnetic radiation. Such a system may include at least a configuration of directed windings, drivers, radio-frequency electromagnetic radiation generators, low-frequency electromagnetic radiation generators, power amplifiers, receiver windings, control electronics, frequency reference, receivers, demodulators, frequency oscillators, digital/analog converters, filters, mixers, preamplifiers, attenuators, an imaging component consisting of receiver windings and EMR coils, receivers, demodulators and acquisition picture, and a measuring component consisting of receiver windings and recorder.

A controller may be utilized to apply the appropriate frequency, duration, intensity, etc., and to drive the currents in windings or coils, which are directed three-dimensionally upon the target tissue directly or in conjunction with a steady magnetic field. The electromagnetic radiation coils can be arranged in different spatial planes with respect to the target tissue to focus the alternating or pulsed electromagnetic radiation field to the preselected target cells. These target cells may be located deep within tissue of the body, or may be located superficially on the body, such as on the skin. Furthermore, in at least one embodiment, the electromagnetic radiation coils are disposed or positioned perpendicular to a central axis of the target cells or tissues. In at least one other embodiment, the electromagnetic radiation coils are disposed or positioned substantially parallel to the central axis of the target cells or tissue.

The effectiveness of the electromagnetic radiation coils can also be enhanced in a variety of ways. For example, a ferromagnetic material can be placed around at least one electromagnetic radiation coil to increase the effectiveness of the electromagnetic radiation field that coil generates. In another embodiment, at least one electromagnetic radiation coil is superconducting, thereby increasing the effectiveness of the field generated thereby. Moreover, the electromagnetic radiation field produced by an electromagnetic radiation coil can be focused and intensified by placing magnetic or paramagnetic material within or surrounding the target are of the body. In one embodiment, the magnetic or paramagnetic material can be placed in or near the target area by injection of the material into nearby blood vessels. In another embodiment, the magnetic or paramagnetic material is placed externally to the target region, such as with an external magnetic device.

The frequency range of the electromagnetic radiation coils can be radio frequency or low frequency. In the case of LF electromagnetic radiation coils, the electromagnetic radiation field can produce polarizations of membranes in different target regions of the body of a patient depending on the size, orientation and geometry of the electromagnetic radiation coil with respect to the target area, the strength of the resultant alternating electromagnetic radiation field, and a frequency with respect to the target area of an alternating electromagnetic radiation field.

In the case of RF electromagnetic radiation coils, the electromagnetic radiation field can produce activation or deactivation of the electron transport system or electrons or protons of nuclei in cancerous cells in the target area depending on at least a size, orientation and geometry of the electromagnetic radiation coil, the intensity of the electromagnetic radiation field, and a frequency with respect to the target area of the steady state electromagnetic radiation field.

The controller can further direct the frequency, duration and magnitude field in the radio frequency range. The Larmor frequency is the reference for the resonant frequency of the target nuclei. A magnetic field, specifically, a steady-state magnetic field produced by a fixed magnet can be utilized in conjunction with RF electromagnetic radiation. Further, X, Y, and Z directed windings can be used, and refer to one spatial embodiment of the coils in orthogonal, or perpendicular, orientations. A receiver and a demodulator may be collectively employed to measure the excitation durations of nuclei in the cancerous tissue via receiver windings. Accordingly, and as noted above, the present EMR treatment regimen may be utilized both to affect treatment as well as to measure the effectiveness of the same.

### Effectiveness of Electromagnetic Radiation Treatment

The EMR treatment regimen in accordance with the present disclosure comprises both an action and a signal. One of the effects of implementation of the present EMR treatment regimen is to initiate a memory so that effectiveness can increase over time. For memory to be initiated, EMR treatment can be performed in a rapid sequence of signals that correspond to the sequential actions of the control system of the cell. Specifically, the sequential actions resulting from implementation of the present EMR treatment regimen in this manner can begin with the cellular or plasma membrane, mitochondrial membrane, and nuclear membrane microtubules. Although, there are a number of other cellular components involved in the sequence, the effect of the present EMR treatment on the membranes of cancerous cells is sufficient to establish the initiation of the cellular memory. The conformation of a specific membrane protein is a component of the memory, but overall memory corresponds to a dynamic-control system in the living cell. To implement memory, the sequential actions within the control system can be those followed by EMR treatment.

It should be appreciated that implementation of the present EMR treatment regimen can assume a number of different forms, patterns or intensities depending on the nature of the cell, its location in the body, the stage of cancer, etc. More in particular, the electromagnetic radiation field applied, as well as frequency, intensity, pulse, etc., can be preselected in accordance with the nature and location of the cancerous cells within the patient's body. When the present EMR treatment regimen is implemented, the memory of the treated cancerous cell cell can correspond to an integration of control system correction, and repeated treatments can demonstrate increasing effectiveness.

### Effects of Electromagnetic Radiation Treatment

Without being bound by theory, Applicant believes that implementation of the EMR treatment regimen as disclosed herein produces at least the following two results: (1) restoration of the missing component(s) of the cell's normal control system, and/or impeding the activity of the cancerous cell's control system; and, (2) initiating apoptosis in cancerous cells. An essential element of the control system of any cell is energy generation, which occurs primarily in the mitochondria, and apoptosis, which is also primarily initiated in the mitochondria. Without being bound by theory, Applicant believes that, to elicit the aforementioned results, the present EMR treatment regimen acts upon at least one of the following: (1) membranes, including cellular, mitochondrial, and nuclear membranes, and particularly focusing upon the membrane potentials or dipoles; and, (2) molecular or atomic entities, including protons, carbon and nitrogen nuclei, the heme group of cytochrome C, protein electron carriers (such as those of the electron transport chain), ATP synthase, DNA and microtubules.

### Illustrative Electromagnetic Radiation Treatment Regimens

The foregoing presented the details of the mechanics and effects of implementation of an electromagnetic radiation (“EMR”) treatment regimen in accordance with the present disclosure. As such, and with that understanding, illustrative examples of various specific EMR treatment regimens are presented below with reference to the figures presented herewith.

### Electromagnetic Radiation Treatment Regimen

In certain embodiments of the present disclosure, EMR treatment regimen requires identifying a target area on a patient for treatment. This may be based upon visual observation, such as the presence of a tumor, lesion, or other externally visible indication of the presence of virally infected cells. In some embodiments, identifying the target area may require non-invasive techniques such as optical or sonic imaging, or invasive techniques, such as, testing of actual tissues samples, in order to detect the presence of cancerous (and/or virally infected cells) and define a target area. In at least one embodiment, the target area may be identified to include the patient's entire body.

Once the target area has been identified, the EMR treatment regimen includes isolating the target area for exposure to electromagnetic radiation. Isolating the target area may simply comprise positioning of one or more electromagnetic coils in proximity to the target area and/or positioning a first or second plate on opposing sides of the target area. In at least one embodiment, isolating the target area comprises the placement of physical barriers between an electromagnetic radiation source and non-targeted areas of the patient. As one example, a shield, such as a lead shield commonly employed to isolate exposure to x-rays, may be positioned over non-targeted areas of the patient's body. Of course, in the event the target area is identified to comprise the patient's entire body, the step of isolating the target area is not required or performed.

In certain embodiments of the present disclosure, a source of electromagnetic radiation can be selected. Certain embodiments of the present disclosure can best be described in connection with the drawings.

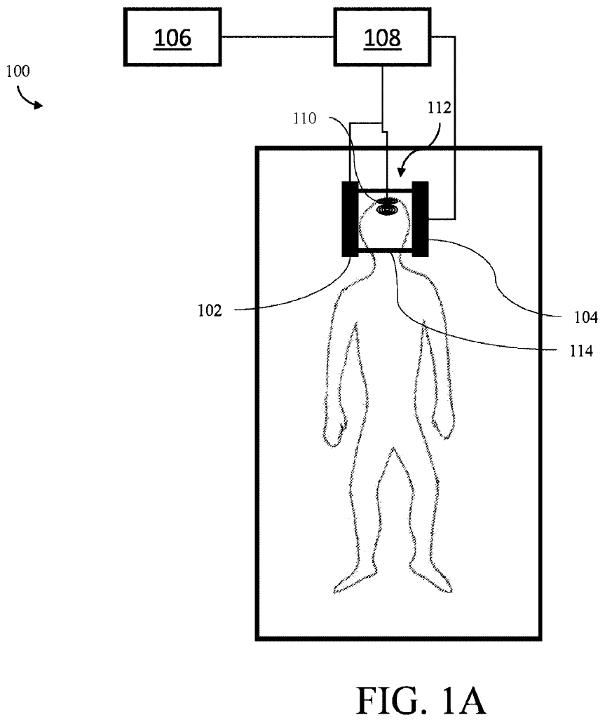

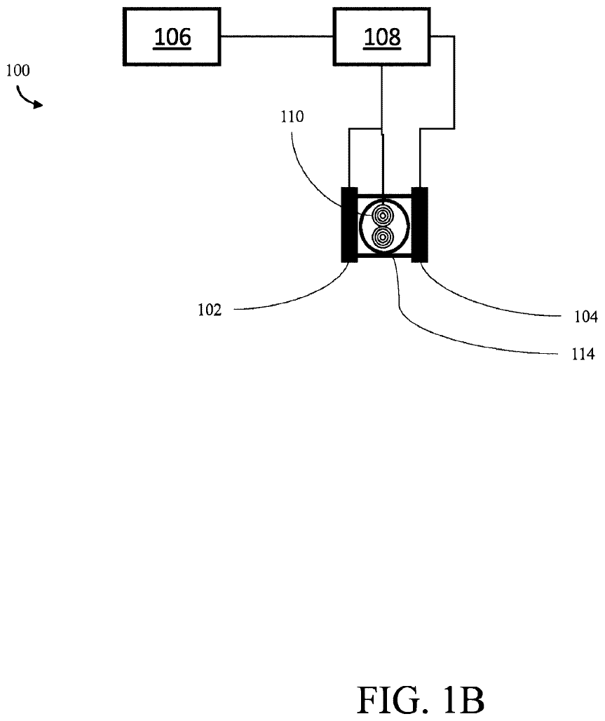

Referring now to the drawings, FIGS. 1A-1B illustrate a system 100 for providing electromagnetic energy (e.g., in connection with treating cancer). System 100 includes a first plate 102 illustrated positioned adjacent to an area 112 to be treated. System 100 also includes a second plate positioned adjacent to the area 112 to be treated and at a distance from first plate 102. Second plate 104 is opposite and not contacting first plate 102. Second plate 104 is substantially parallel to first plate 102. System 100 also includes an electromagnetic coil 110. Electromagnetic coil 110 is configured to receive electrical energy and is illustrated positioned between first plate 102 and second plate 104. Electromagnetic coil 110 can be a “Figure-8” type coil.

System 100 can also include a power source 106. Power source 106 can be configured to supply electrical energy to first plate 102, second plate 104, and electromagnetic coil 110.

System 100 can also include a controller 108 electrically coupled to first plate 102, second plate 104, electromagnetic coil 110, and power source 110. Controller 108 can be configured to selectively provide electrical energy such that a first electromagnetic field is induced between first plate 102 and second plate 104 within the area 112 to be treated. First electromagnetic field can have a frequency range of 50-900 kHz. In some embodiments, the first electromagnetic field can have a frequency of between about 50 kHz to about 100 kHz, between about 100 kHz to about 150 kHz, between about 150 kHz to about 200 kHz, between about 200 kHz to about 250 kHz, between about 250 kHz to about 300 kHz, between about 300 kHz to about 350 kHz, between about 350 kHz to about 400 kHz, between about 400 kHz to about 450 kHz, between about 450 kHz to about 500 kHz, between about 500 kHz to about 550 kHz, between about 550 kHz to about 600 kHz, between about 600 kHz to about 650 kHz, between about 650 kHz to about 700 kHz, between about 700 kHz to about 750 kHz, between about 750 kHz to about 800 kHz, between about 800 kHz to about 850 kHz, or between about 850 kHz to about 900 kHz.

Controller 108 can be configured to selectively provide electrical energy such that a second electromagnetic field is induced by the electromagnetic coil. In certain regards, at least one of the electromagnetic fields interacts with intracellular activities and functions, such as the mitochondria and other cellular-regulation mechanisms.

In certain embodiments, the first electromagnetic field can be defined by a frequency range of 100-300 kHz; 150-250 kHz; 180-220 kHz; 190-210 kHz; 195-205 kHz; and 200 kHz. In some embodiments, the first electromagnetic field can be defined by a frequency range of about 100 kHz to about 110 kHz, about 110 kHz to about 120 kHz, about 120 kHz to about 130 kHz, about 130 kHz to about 140 kHz, about 140 kHz to about 150 kHz, about 150 kHz to about 160 kHz, about 160 kHz to about 170 kHz, about 170 kHz to about 180 kHz, about 180 kHz to about 190 kHz, about 190 kHz to about 200 kHz, about 200 kHz to about 210 kHz, about 210 kHz to about 220 kHz, about 220 kHz to about 230 kHz, about 230 kHz to about 240 kHz, about 240 kHz to about 250 kHz, about 250 kHz to about 260 kHz, about 260 kHz to about 270 kHz, about 270 kHz to about 280 kHz, about 280 kHz to about 290 kHz, or about 290 kHz to about 300 kHz.

In certain embodiments, the first electromagnetic field can be defined by a voltage range of 18 V-300 V. In some embodiments, the first electromagnetic field can be defined by a voltage of between about 15 V to about 25 V, between about 25 V to about 35 V, between about 35 V to about 45 V, between about 45 V to about 55 V, between about 55 V to about 65 V, between about 65 V to about 75 V, between about 75 V to about 85 V, between about 85 V to about 95 V, between about 95 V to about 105 V, between about 105 V to about 115 V, between about 115 V to about 125 V, between about 125 V to about 135 V, between about 135 V to about 145 V, between about 145 V to about 155 V, between about 155 V to about 165 V, between about 165 V to about 175 V, between about 175 V to about 185 V, between about 185 V to about 195 V, between about 195 V to about 205 V, between about 205 V to about 215 V, between about 215 V to about 225 V, between about 225 V to about 235 V, between about 235 V to about 245 V, between about 245 V to about 255 V, between about 255 V to about 265 V, between about 265 V to about 275 V, between about 275 V to about 285 V, between about 285 V to about 295 V, or between about 295 V to about 305 V.

In certain embodiments, the electromagnetic coil can be configured and adapted to provide the second electromagnetic field at a frequency to induce resonance of ions or molecules of a cell, a neuron, or a membrane within the area to be treated. For example, second electromagnetic field can be configured to target specific ions (e.g., protons) or molecules in the membrane (or adjacent to it) that have a precise Larmor frequency to achieve resonance. The electromagnetic coil can be configured to provide the second electromagnetic field having a frequency range of 10-60 megahertz (“MHz”). In some embodiments, the electromagnetic coil can be configured to provide the second electromagnetic field having a frequency range of between about 10 MHz to about 15 MHz, between about 15 MHz to about 20 MHz, between about 20 MHz to about 25 MHz, between about 25 MHz to about 30 MHz, between about 30 MHz to about 35 MHz, between about 35 MHz to about 40 MHz, between about 40 MHz to about 45 MHz, between about 45 MHz to about 50 MHz, between about 50 MHz to about 55 MHz, or between about 55 MHz to about 60 MHz. In some embodiments, the Electromagnetic coil 110 can be controlled independently from the first plate and the second plate.

In certain embodiments, electromagnetic coil 110 can define a shape and a diameter. Electromagnetic coil 110 can be configured and adapted to change the shape or the diameter to treat the area to be treated. For example, electromagnetic coil 110 can be a Figure-8 coil, as illustrated in FIG. 1B. In another example, the conductive structures (e.g., conductive structures, copper, wires, and the like) of electromagnetic coil 110 can be manipulated (e.g., elongated, squeezed, bent, etc.) to change the orientation or magnitude of the generated electromagnetic field.

In certain embodiments, first electromagnetic field and the second electromagnetic field can be provided for a cumulative duration for, for example: 5 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 24 hours, 48 hours, and 72 hours. In certain embodiments, the cumulative duration could be a combination of LF electromagnetic radiation and RF electromagnetic radiation. For example, a cumulative duration of 60 minutes could mean 30 minutes of LF electromagnetic radiation and 30 minutes of RF electromagnetic radiation. In certain embodiments, a cumulative duration of 60 minutes could mean 50 minutes of LF electromagnetic radiation and 10 minutes of RF electromagnetic radiation. In certain embodiments, a cumulative duration of 60 minutes could mean 40 minutes of LF electromagnetic radiation and 20 minutes of RF electromagnetic radiation. In certain embodiments, a cumulative duration of 60 minutes could mean 20 minutes of LF electromagnetic radiation and 40 minutes of RF electromagnetic radiation. In certain embodiments, a cumulative duration of 60 minutes could mean 10 minutes of LF electromagnetic radiation and 50 minutes of RF electromagnetic radiation. The cumulative duration could be split between LF electromagnetic radiation and RF electromagnetic radiation in any way to total the cumulative duration. In certain embodiments, the cumulative duration could be either of LF electromagnetic radiation or RF electromagnetic radiation. For example, a cumulative duration of 60 minutes could mean 60 minutes of LF electromagnetic radiation and 0 minutes of RF electromagnetic radiation, or vice versa.

In certain embodiments, a spacing system 114 can be used to space first plate 102 and second plate 104. Spacing system 114 can be configured to position each of first plate 102, second plate 104, and electromagnetic coil 110 with respect to area 112 to be treated. Spacing system 114 can include a plurality of rails configured to support first plate 102 and/or second plate 104 and further configured to maintain a parallelism of between first plate 102 and 104. In some embodiments, the distance between the first plate 102 and second plate 104 can be about 5 in., about 6 in., about 7 in., about 8 in., about 9 in., about 10 in., about 11 in., about 12 in., about 13 in., about 14 in., about 15 in., about 16 in., about 17 in., about 18 in., about 19 in., or about 20 in.

In some embodiments, the first plate 102 and second plate 104 could be spaced such that the electromagnetic field induced between the first plate 102 and the second plate 104 is a central distance between the first plate 102 and second plate 104. In some embodiments, the first plate 102 and second plate 104 are spaced such that the tumor to be treated is centrally located between the plates. In some embodiments, the first plate 102 and second plate 104 are spaced such that the electromagnetic field is centrally positioned on the tumor.

In some embodiments, the size and strength of the electromagnetic field induced between the first plate 102 and the second plate 104 is a function of the distance that the first plate 102 and second plate 104 are spaced. In some embodiments, the farther the distance between the first plate 102 and second plate 104, the larger the electromagnetic field, and the lower the impact of the electromagnetic field on the tumor. In some embodiments, the shorter the distance between the first plate 102 and second plate 104, the smaller the electromagnetic field, and the greater the impact of the electromagnetic field on the tumor. The size and strength of the electromagnetic field can be adjusted and controlled based on the spacing between the first plate 102 and the second plate 104.

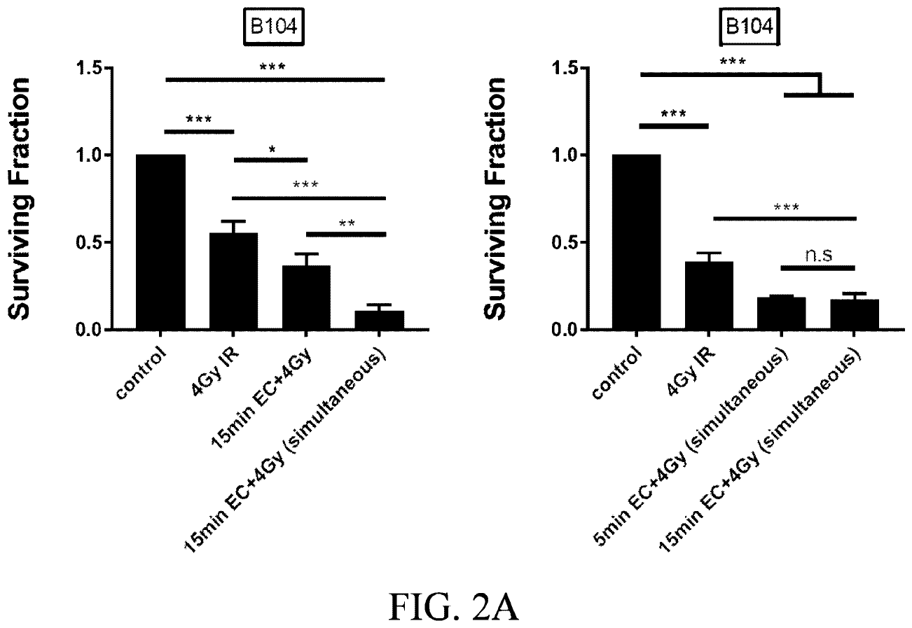

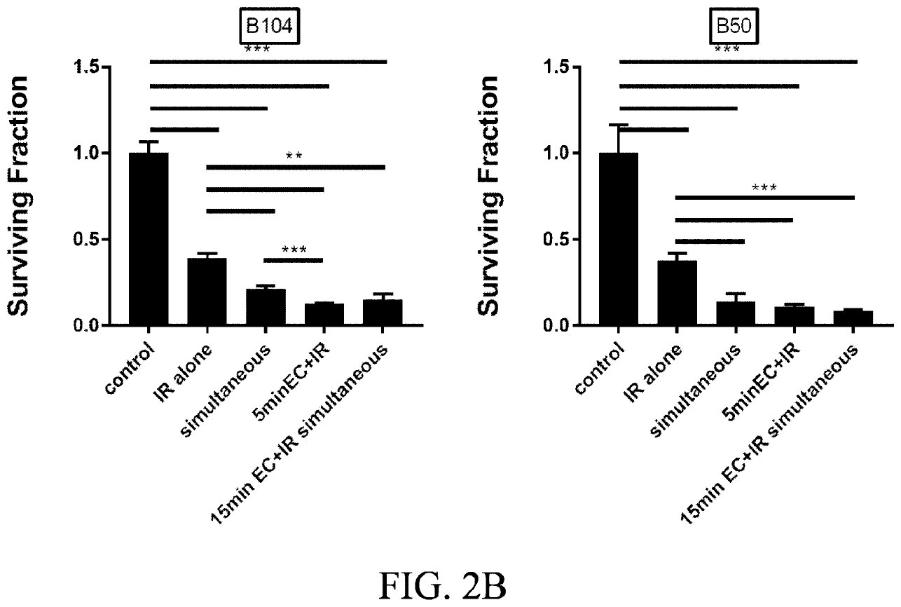

FIGS. 2A-2B illustrate graphs of the surviving fraction on a cell culture preparation (e.g., glioblastoma sample from a patient provided by a neurosurgeon) in response to various treatments. For the sample labelled “4Gy IR”, ionizing radiation was applied (i.e., a standard application and standard treatment). For the samples labelled “15 min EC+4GY”, electromagnetic radiation of certain embodiments of the present disclosure was applied prior to ionizing radiation (i.e., done sequentially). For the samples labelled “15 min EC+4GY (simultaneous)”, electromagnetic radiation of certain embodiments of the present disclosure was applied for 15 minutes simultaneously with ionizing radiation. For the samples labelled “5 min EC+4GY (simultaneous)”, electromagnetic radiation of certain embodiments of the present disclosure was applied for 5 minutes simultaneously with ionizing radiation. The titles of each graph indicate the neuroblastoma B50 and B104 cells culture used.

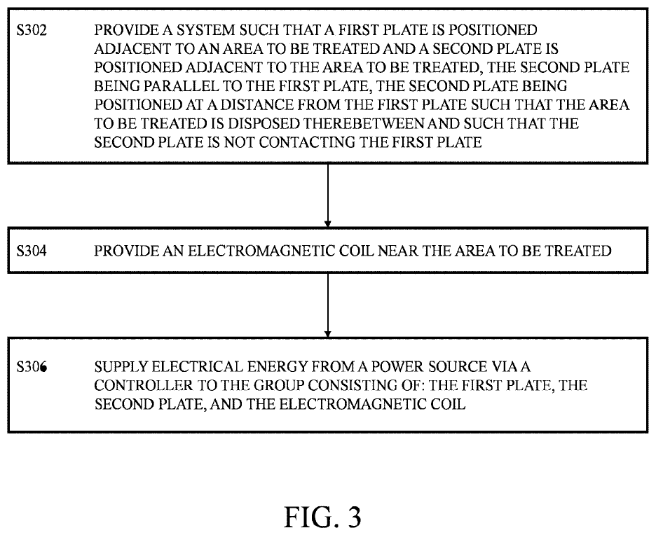

FIG. 3 is a flow diagram in accordance with exemplary embodiments of the present disclosure. As is understood by those skilled in the art, certain steps included in the flow diagrams may be omitted; certain additional steps may be added; and the order of the steps may be altered from the order illustrated.

FIG. 3 illustrates a method of providing electromagnetic energy using the certain systems described herein. At step S302, a system is provided such that a first plate is positioned adjacent to an area to be treated and a second plate is positioned adjacent to the area to be treated, the second plate being substantially parallel to the first plate, the second plate being positioned at a distance from the first plate such that the area to be treated is disposed therebetween and such that the second plate is not contacting the first plate. At S304, an electromagnetic coil is provided near the area to be treated. At S306, supply electrical energy from a power source via a controller to the group consisting of: the first plate, the second plate, and the electromagnetic coil.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A system for providing electromagnetic energy comprising:
a means for applying ionizing radiation to the area to be treated;
a first plate configured to be positioned adjacent to an area to be treated;
a second plate configured to be positioned adjacent to the area to be treated and at a distance from the first plate, the second plate being opposite and not contacting the first plate, the second plate being configured to be substantially parallel to the first plate;
an electromagnetic coil configured to receive electrical energy;
a power source configured to supply electrical energy to the first plate, the second plate, and the electromagnetic coil; and
a controller electrically coupled to one or more of the means for applying ionizing radiation, the first plate, the second plate, the electromagnetic coil, and the power source, the controller being configured to independently provide electrical energy such that:
ionizing radiation is generated by the means for applying ionizing radiation;
a first electromagnetic field is induced between the first plate and the second plate within the area to be treated, wherein the first electromagnetic field has a frequency range of 50-900 kHz; and
a second electromagnetic field is induced by the electromagnetic coil.

2. The system of claim 1, further comprising a spacing system, the spacing system being configured to position each of the first plate, the second plate, and the electromagnetic coil with respect to the area to be treated.

3. The system of claim 1, wherein the first electromagnetic field is defined by a frequency range of 100-300 kHz.

4. The system of claim 1, wherein the first electromagnetic field is defined by a frequency of 200 kHz.

5. The system of claim 1, wherein the first electromagnetic field is defined by a voltage range of 18-300 V.

6. The system of claim 1, wherein the electromagnetic coil is configured and adapted to provide the second electromagnetic field at a frequency to induce resonance of ions or molecules of a cell, a neuron, or a membrane within the area to be treated.

7. The system of claim 1, wherein the electromagnetic coil is configured to provide the second electromagnetic field having a frequency range of 10-60 MHz.

8. The system of claim 1, wherein the electromagnetic coil is controlled independently from the first plate and the second plate.

9. The system of claim 1, wherein the electromagnetic coil defines a shape and a diameter, wherein the electromagnetic coil is configured and adapted to change the shape or the diameter to treat the area to be treated.

10. The system of claim 1, wherein the electromagnetic coil is a Figure-8 coil.

11. A method of providing electromagnetic energy using the system of claim 1, comprising the steps of:
a) providing the system such that the means for applying ionizing radiation is positioned near the area to be treated, the first plate is positioned adjacent to the area to be treated and the second plate is positioned adjacent to the area to be treated, the second plate being substantially parallel to the first plate, the second plate being positioned at a distance from the first plate such that the area to be treated is disposed therebetween and such that the second plate is not contacting the first plate;
b) providing the electromagnetic coil near the area to be treated;
c) supplying electrical energy from the power source via the controller to the group consisting of: the first plate, the second plate, and the electromagnetic coil; and
d) treating the area to be treated with ionizing radiation.

12. The method of claim 11, further comprising wherein:
a first electromagnetic field is induced between the first plate and the second plate within the area to be treated, wherein the first electromagnetic field has a frequency range of 50-900 kHz; and
a second electromagnetic field is induced by the electromagnetic coil, wherein the second electromagnetic field has a frequency range of 10-60 MHz.

13. The method of claim 11, wherein the first electromagnetic field is defined by a frequency range of 100-300 kHz.

14. (canceled)

15. The method of claim 11, wherein the first electromagnetic field and the second electromagnetic field are provided for a cumulative duration selected from the group consisting of: 5 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 24 hours, 48 hours, and 72 hours.

16. The method of claim 11, wherein the supplying of step (c) and the treating of step (d) are done simultaneously for up to 5 minutes.

17. The method of claim 11, wherein the supplying of step (c) and the treating of step (d) are done simultaneously for up to 15 minutes.

18. The method of claim 11, wherein during step (a):
the first plate is provided to a left section of the area to be treated; and
the second plate is provided to a right section of the area to be treated; and
wherein during step (b), the electromagnetic coil is provided to a frontal section of the area to be treated.

19. The method of claim 11, wherein step (c) includes supplying electrical energy with a first characteristic to the electromagnetic coil independently from the electrical energy with a second characteristic supplied to the first plate and second plate, wherein the first characteristic is defined by a first frequency and a first power, wherein the second characteristic is defined by a second frequency and a second power.

20. The method of claim 11, further comprising the step of:
(f) configuring a shape or a diameter of the electromagnetic coil such that the electromagnetic coil is configured and adapted to treat the area to be treated.

21. The method of claim 20, wherein the configuring of step (f) changes a focus and an intensity of the second electromagnetic field.
